(19) United States
(12) Patent Application Publication
(10) Pub. No.: US 2022/0273558 A1
(43) Pub. Date: Sep. 1, 2022

(54) Chewing Gum Compositions Containing Cannabinoids

(71) Applicant: ADVANCED FEMALE TECHNOLOGIES LLC, New York, NY (US)

(72) Inventor: Albert Feinstein, New York, NY (US)

(21) Appl. No.: 17/632,437

(22) PCT Filed: Jul. 31, 2020
(86) PCT No.: PCT/US20/70348

§ 371 Date: Feb. 2, 2022

Related U.S. Application Data
(60) Provisional application No. 62/882,253, filed on Aug. 2, 2019.

(51) Int. Cl. A61K 9/68; A61K 31/195; A61K 31/53; A61K 31/5513; A61K 31/4015; A61K 31/19; A61K 31/55; A61K 31/197; A61K 31/36; A61K 31/436; A61K 31/4192; A61K 31/138; A61K 31/165; A61K 31/5517; A61K 31/05; A61K 31/352; A61K 47/02; A61K 47/24; A61K 47/44

(52) U.S. Cl. CPC A61K 9/0058; A61K 31/195; A61K 31/53; A61K 31/5513; A61K 31/4015; A61K 31/19; A61K 31/55; A61K 31/197; A61K 31/36; A61K 31/436; A61K 31/4192; A61K 31/138; A61K 31/165; A61K 31/5517; A61K 31/05; A61K 31/352; A61K 47/02; A61K 47/24; A61K 47/44

(57) ABSTRACT

This disclosure provides a chewing gum composition comprising one or more cannabinoids, and/or derivatives thereof. The chewing gum compositions may additional contain one or more anti-epileptic drugs (AEDs) or one or more anti-tremor dmgs. The chewing gum composition is formulated to provide controlled release of the active agents during mastication. Methods to treat tremors or seizures using the chewing gum composition are also provided.

### CROSS-REFERENCE TO RELATED APPLICATION

This application claims the benefit of U.S. Provisional Application No. 62/882,253, filed Aug. 2, 2019, which is incorporated herein by reference in its entirety.

### FIELD OF THE INVENTION

A chewing gum composition comprising one or more cannabinoids, and/or derivatives thereof, and one or more anti-epileptic drugs (AEDs) or anti-tremor drugs is provided. The chewing gum composition is formulated to provide release of the one or more cannabinoid and the AED or anti-tremor drugs during mastication. Methods to treat tremors or seizures using the chewing gum compositions are also provided.

### BACKGROUND

Approximately 1% of the population worldwide suffers from epilepsy of which 70% are able to obtain seizure freedom with existing anti-epileptic drugs (AEDs). However, 30% of this patient group are unable to control symptoms with the AEDs that are available and as such are termed as suffering from intractable or “treatment-resistant epilepsy” (TRE).

Several neurological diseases or disorders are associated with tremor. Parkinson's disease, for example, is a neurological disorder that leads to shaking, stiffness, and difficulty with walking, balance, and coordination. It occurs when nerve cells, or neurons, in an area of the brain that controls movement become impaired and/or die, and is caused, in part, by a deficiency of the neurotransmitter, dopamine. Available drugs fail to halt progression of various symptoms of Parkinson's disease such as tremor, rigidity, and progressive dementia.

There is a need for compositions to treat seizure, in particular for patients who have already failed to respond to AEDs. In addition, there is a need for compositions to treat tremor, in particular for patients having tremor that does not respond to other therapies.

### SUMMARY OF THE INVENTION

The present disclosure relates to the use of a chewing gum composition containing one or more cannabinoids in the treatment of seizures or tremors. The present disclosure further relates to the use of a chewing gum composition containing one or more cannabinoids in the treatment of epilepsy or neurological diseases or disorders.

In one aspect, the present disclosure provides a chewing gum delivery system for one or more cannabinoids. In embodiments, the chewing gum further comprises one or more AEDs. In embodiments, the chewing gum further comprises one or more anti-tremor agents. The active ingredients may be released in a controlled manner for use in the treatment of seizures (including those associated with epilepsy) or in the treatment of tremors (including those associated with neurological diseases or disorders). The chewing gum composition is a delivery system which provides controlled release of the active pharmaceutical ingredients and dual action mechanism for treatment of the conditions disclosed herein. A chewing gum delivery system provides a much faster than conventional ingestible orally delivered products like capsules, pills or liquid because active ingredients are provided in a transmucosally absorbable form.

In one aspect, the disclosure provides a chewing gum composition comprising one or more cannabinoids, one or more anti-epileptic drugs (AEDs) and a gum base. In one aspect, the disclosure provides a chewing gum composition comprising one or more cannabinoids, one or more anti-tremor drugs and a gum base. In embodiments, the one or more cannabinoids is selected from the group consisting of cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof. In embodiments, the one or more cannabinoids is or comprises CBD.

In embodiments, the chewing gum composition may comprise anti-epileptic drugs AEDs such as rufinamide, lamotrigine, felbamate, clobazam and topiramate, carbamezapine, gabapentin, oxcarbazepine, pregabalin, tiagabineor and vigabatrin, acetazolamide, brivaracetam, clonazepam, eslicarbazepine acetate, ethosuximide, everolimus, lacosamide, lamotrigine, levetiracetam, oxcarbazepine, perampanel, phenobarbital, phenytoin, piracetam, pregabalin, primidone, rufinamide, sodium valproate, stiripentol, tiagabine, topiramate, valproic acid, vigabatrin, zonisamide or combinations thereof. In embodiments, the one or more AEDs is selected from the group consisting of as rufinamide, lamotrigine, felbamate, clobazam and topiramate, carbamezapine, gabapentin, oxcarbazepine, pregabalin, tiagabineor and vigabatrin, and combinations thereof. In embodiments, the AED is or comprises gababentin.

In another aspect, the disclosure provides a method of treating seizures comprising administering to a subject in need thereof, a gum-based composition for chewing as described in the previous paragraphs and/or as described herein. In embodiments, the seizure is associated with epilepsy. In embodiments, the seizures are associated with Lennox-Gastaut syndrome (LGS), Dravet syndrome, self-limited, late-onset, occipital epilepsy (Gastaut syndrome), epilepsy with eyelid myoclonia (Jeavons syndrome), temporal lobe epilepsy (TLE), juvenile absence epilepsy (JAE) or juvenile myoclonic epilepsy (JME). In embodiments, the epilepsy is Lennox-Gastaut syndrome. In embodiments, the subject in need thereof has not responded to one or more AED drugs. In embodiments, the method results in a reduction of the frequency of drop seizures and/or the reduction in the frequency of total seizures.

In embodiments, the chewing gum composition comprises one or more anti-tremor drugs. In embodiments, the one or more anti-tremor drugs is an AED, for example, primidone, topiramate, zonisamide, pregabalin, and/or gabapentin. In embodiments, the one or more anti-tremor drugs is a beta blocker, a benzodiazepine, or combinations thereof. In embodiments, the one or more anti-tremor drugs is selected from the group consisting of propranolol, atenolol, metoprolol, nadolol, sotalol, alprazolam, clonazepam, diazepam, and lorazepam, or combinations thereof.

In another aspect, the disclosure provides a method of treating tremor in a subject in need thereof by administering a chewing gum composition comprising one or more cannabinoids and a gum base. In embodiments, the one or more cannabinoids comprises CBD. In further embodiments, the chewing gum composition further comprises THC. In embodiments the chewing gum composition comprises an anti-tremor drug, including those disclosed herein. In embodiments, the tremor is associated with a neurological disease or disorder. In embodiments, the neurological disease or disorder is multiple sclerosis, stroke, traumatic brain injury, Alzheimer's disease, Parkinson's disease, Huntington chorea; amyotrophic lateral sclerosis (ALS); Tourette syndrome; Pick's disease; multiple system atrophy (MSA), dementia with Lewy bodies (DLB), progressive supranuclear palsy (PSP), or corticobasal ganglionic degeneration (CBGD). In embodiments, the neurological disease is multiple sclerosis or Parkinson's disease. In embodiments, the tremor is essential tremor; dystonic tremor, cerebellar tremor, psychogenic tremor, or physiologic tremor. In embodiments, the tremor is associated with a hyperkinetic movement including, but not limited to, chorea, dystonia, athetosis, myoclonus, stereotypies, tics, and hemiballismus

In embodiments, the gum base comprises one or more elastomers, resins, softening agents, emulsifiers and/or fillers. In embodiments, the gum base comprises couma macrocarpa, glycerol esters of gum, microcrystalline wax, lecithin and calcium carbonate.

In embodiments, the chewing gum composition further comprises one or more sugar alcohols. In further embodiments, the sugar alcohol is maltitol, sorbitol, isomalt, and/or xylitol. The chewing gum compositions may also contain one or more additional sweeteners. In embodiments, the additional sweeteners are selected from a sugar, a high intensity sweetener, a sugar substitute, or a combination thereof. In a further embodiment, the sugar substitute is Stevia.

The chewing gum compositions may also contain one or more flavoring agents, and one or more lubricants and powder flow agents. In embodiments, the flavoring agent is a mint flavoring including, e.g., peppermint oil, oil of wintergreen or spearmint oil. In embodiments, the lubricants and powder flow agents are magnesium stearate, and silicon dioxide.

In embodiments, the chewing gum composition comprises one or more sugar alcohols, a gum base, one or more flavoring agents, one or more sweeteners, one or more cannabinoids, and tableting excipients. In further embodiments, the composition comprises about 45-65% sugar alcohols, about 15-35% gum base, about 2-15% flavoring agents and/or sweeteners, and about 1-10% tableting excipients.

In embodiments, the chewing gum compositions described herein may be manufactured by a method comprising the steps of:
(i) placing a gum base in a container with a sugar alcohol and heating to an internally-measured temperature of between 140-160° F. to melt the gum base;
(ii) combining the one or more cannabinoids and one or more AEDs, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).

### DETAILED DESCRIPTION OF THE INVENTION

The present disclosure provides a chewing gum composition comprising a gum base and one or more cannabinoids. The chewing gum compositions may further comprise an AED and/or anti-tremor drug. The chewing gum composition described herein provides faster release of cannabinoid (and other active agents if present) providing onset of action in about 5 to about 10 minutes, or less, as compared to orally administered dosage forms like tablets and capsules, which can take up to 40 minutes to absorb through the digestive tract.

Due to the high density of blood vessels in the buccal mucosa, the chewing gum compositions described herein “buccally” deliver one or more cannabinoids resulting in direct access to the systemic circulation, avoiding first-pass hepatic metabolism, and avoiding exposure to the acidic environment of the gastrointestinal tract. In addition, the buccal mucosa has low enzymatic activity relative to the nasal and rectal routes. Thus, the potential for inactivation of buccally delivered cannabinoids due to biochemical degradation is less extensive than other administration routes.

During the chewing process, the active agent or agents directly enter the blood stream through the oral mucosa (buccal or sublingual) allowing for fast alleviation of symptoms. The absorption through the oral mucosa is up to five times faster than conventional ingestible orally delivered products such as capsules, pills or liquids because it bypasses the digestive system and directly enters the bloodstream. Thus, the present disclosure provides a chewing gum delivery system that provides a therapeutically effective concentration of a cannabinoid (e.g., CBD) in the bloodstream within about 5 to about 10 minutes of administration.

The chewing gum composition described provides one or more cannabinoid (e.g., CBD) in a transmucosally-absorbable form. Absorption from the gum formulation occurs primarily through the buccal mucosa, which increases the rate of absorption of the active ingredients. Cannabinoids administered in the chewing gum formulation are absorbed at a significantly faster rate, while bioavailability is comparable to, or greater than, that of a capsule formulation. As the onset of action for the gum delivery is within 5 to 10 minutes, or less, of administration, the dose of cannabinoid can be quickly and easily titrated. Consequently the chewing gum composition described is a convenient and effective means of rapidly administering one or more cannabinoids, and is useful for the treatment of seizures and the treatment of tremors.

The present disclosure provides methods of treating seizures in a subject in need thereof and methods of treating tremors in a subject in need thereof by administering the chewing gum compositions described herein. Administering as used herein means directing to take or taking (e.g., placing in the mouth and chewing) the chewing gum compositions described herein.

Cannabis Extract

In embodiments, the chewing gum composition of the present disclosure comprises a cannabinoid plant extract. The cannabis plant has many naturally occurring substances that are of medical interest. Isolated compounds from the cannabis plant include 19-tetrahydrocannabinol (THC), cannabidiol (CBD), cannabichromene (CBC), cannabigerol (CBG), cannabidivarin (CBDV), among other compounds. There are a total of one hundred and forty one (141) cannabinoids that have been isolated from the cannabis plant. The term “extract” as used herein is a preparation containing one or more active ingredients (e.g., one or more cannabinoids) derived from plant material. Extraction methods include, for example, cold extraction techniques, soxhlet extraction, decoction, supercritical extraction, microwave extraction, and/or extraction using one or more extraction solvents including, but not limited to, ethanol, methanol, acetone, acetonitrile, chloroform, water, hydroalcoholic mixture, and isopropyl alcohol.

Cannabinoids

Cannabinoid are a class of chemical compounds that acts on cannabinoid receptors, and include compounds found in cannabis, endogenous compounds, and synthetic cannabinoids. Examples of cannabinoids include, but are not limited to, cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and tetrahydrocannabinol (THC). While THC has psychoactive effects, CBD, CBC, CBG, and CBDV, for example, do not. Cannabinoids described in the present application include, but are not limited to, those listed below along with their standard abbreviations and chemical structure.

| | | |
|---|---|---|
| CBD | Cannabidiol | 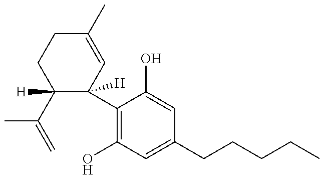 [SMILES: C=C(C)[C@@H]1CCC(C)=C[C@H]1c1c(O)cc(CCCCC)cc1O] |
| CBDA | Cannabidiolic acid | 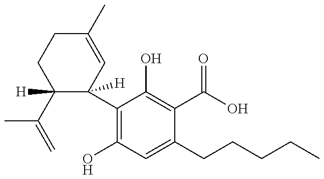 [SMILES: C=C(C)[C@@H]1CCC(C)=C[C@H]1c1c(O)cc(CCCCC)c(C(=O)O)c1O] |
| CBDV | Cannabidivarin | 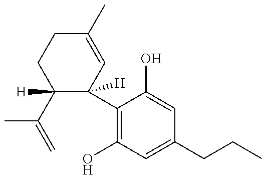 [SMILES: C=C(C)[C@@H]1CCC(C)=C[C@H]1c1c(O)cc(CCC)cc1O] |
| CBDVA | Cannabidivarinic acid | 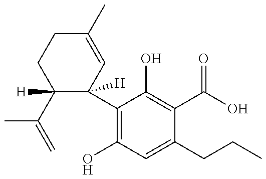 [SMILES: C=C(C)[C@@H]1CCC(C)=C[C@H]1c1c(O)cc(CCC)c(C(=O)O)c1O] |
| THC | Tetrahydrocannabinol | 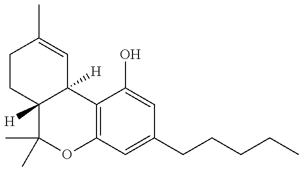 [SMILES: CCCCCc1cc(O)c2c(c1)OC(C)(C)[C@@H]1CCC(C)=C[C@@H]21] |

In embodiments, the chewing gum composition comprises CBD, another cannabinoid, combinations of CBD and other cannabinoids, or combinations of other cannabinoids. Preferably the CBD used is in the form of a highly purified extract of cannabis such that the CBD is present at greater than 98% of the total extract (w/w). In embodiments, the cannabinoids (other than CBD) of the extract are characterized. In embodiments, the cannabinoid tetrahydrocannabinol (THC) has been substantially removed, to a level of not more than 0.15% (w/w) and/or the propyl analogue of CBD, cannabidivarin, (CBDV) is present in amounts of up to 1%. In embodiments, the cannabis extract comprises less than about 0.10%, 0.05%, or 0.01% THC (w/w). In embodiments, the extract comprises no more than 0.15% CBDA and/or no more than 0.5% CBD-C4. Alternatively, the CBD may be a synthetically produced CBD.

In embodiments, the cannabinoid used in the chewing gum composition is obtained as a liquid carbon dioxide extract of high-CBD containing varieties of Cannabis sativa L., which has been further purified by a solvent crystallization method to yield CBD. The crystallization process specifically removes other cannabinoids and plant components to yield greater than 98% CBD. Although the CBD is highly purified, because it is produced from a cannabis plant rather than synthetically there are other cannabinoids which are co-produced and co-extracted with the CBD. Similar methods for extracting cannabinoids other than CBD are also known in the art.

In embodiments, the chewing gum composition comprises about 2 to about 50 or more mg of a cannabis extract. In embodiments, the chewing gum composition comprises about 0.1 to about 50 or more mg of one or more cannabinoids. In embodiments, the chewing gum composition comprises about 0.1 to about 90 mg of CBD, for example, about 0.1 mg to about 5 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, about 40 mg to about 50 mg, about 45 mg to about 55 mg, about 50 mg to about 60 mg, about 55 mg to about 65 mg, about 60 mg to about 70 mg, about 65 mg to about 75 mg, about 70 mg to about 80 mg, about 75 mg to about 85 mg, or about 80 mg to about 90 mg.

In embodiments, the chewing gum composition comprises CBD and THC. In such embodiments, the CBD may be present in about 0.1-50 mg, or more, and the THC may be present in about 0.1-50 mg, or more, for example CBD and THC are independently present in about 0.1 mg to about 10 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, or about 40 mg to about 50 mg.

Anti-Epileptic Drugs (AEDs)

In embodiments, the chewing gum composition may comprise anti-epileptic drugs AEDs such as rufinamide, lamotrigine, felbamate, clobazam and topiramate, carbamezapine, gabapentin, oxcarbazepine, pregabalin, tiagabineor and vigabatrin, acetazolamide, brivaracetam, clonazepam, eslicarbazepine acetate, ethosuximide, everolimus, lacosamide, lamotrigine, levetiracetam, oxcarbazepine, perampanel, phenobarbital, phenytoin, piracetam, pregabalin, primidone, rufinamide, sodium valproate, stiripentol, tiagabine, topiramate, valproic acid, vigabatrin, zonisamide or combinations thereof. AEDs such as sodium valproate may be used in combination with rufinamide or lamotrigine. In embodiments, the AED is felbamate, clobazam or topiramate. In embodiments, certain AEDs such carbamezapine, gabapentin, oxcarbazepine, pregabalin, tiagabineor and vigabatrin are contraindicated in treating drop seizures.

Typical doses for these AEDs in treating seizures are known in the art. In embodiments, the dose of AED is reduced by 5%, 10%, 20%, 30%, 40%, 50%, or more, from standard dosing amounts when administered along with a composition comprising CBD, as serum levels of AEDs can increase in the presence of CBD.

Anti-Tremor Drugs

In embodiments, the chewing gum composition comprises one or more anti-tremor drugs (i.e., a drug used to treat tremor). In embodiments, the anti-tremor drug is an AED listed above, for example, primidone, topiramate, zonisamide, pregabalin, and gabapentin. In embodiments, the anti-tremor drug is a beta-adrenergic blocking agent (beta blocker) such as propranolol, atenolol, metoprolol, nadolol, and sotalol. In embodiments, the anti-tremor drug is a benzodiazepine such as alprazolam, clonazepam, diazepam, and lorazepam.

Methods of Treating Seizures

In accordance with one aspect of the present disclosure there is provided chewing gum compositions containing one or more cannabinoids for the treatment of seizures. In embodiments, the seizures are associated with epilepsy. Clinical trials on patients with epilepsy indicate that the use of CBD in combination with AEDs provides a reduction in both drop seizures and total seizure frequency. In embodiments, the cannabinoid comprises, consists of, or consists essentially of CBD. In embodiments, the method provides treating seizures associated with Lennox-Gastaut syndrome (LGS), Dravet syndrome, self-limited, late-onset, occipital epilepsy (Gastaut syndrome), epilepsy with eyelid myoclonia (Jeavons syndrome), temporal lobe epilepsy (TLE), juvenile absence epilepsy (JAE) or juvenile myoclonic epilepsy (JME). In further embodiments, the method is characterized in that the patient has not responded to one or more anti-epileptic drugs (AEDs). In embodiments, administration of a chewing gum composition described herein provides significant reduction in both drop seizure and total seizure frequency.

Treatment of seizures is accomplished by administering the chewing gum composition disclosed herein to a subject in need thereof. The term treatment is used in its broadest sense and, as used herein, means lessening the severity, lessening the frequency, or otherwise alleviating seizures.

The method of treating seizures comprises chewing one or two pieces of gum comprising one or more cannabinoids as described herein. In embodiments, the chewing gum composition further comprises one or more AEDs as described herein. Typically, up to two pieces of the chewing gum composition maybe chewed at one time. Cannabinoids and derivatives thereof and AEDs in these chewing gums according to embodiments may be released in a controlled manner and absorbed by a subject via transmucosal delivery mechanism. A mammal, such as a human being, may chew the chewing gum composition according to embodiments described herein 1 to 6 times a day to reduce the frequency of seizures and/or total seizures.

Preferably the CBD is in the form of a highly purified extract of cannabis which comprises at least 98% (w/w) CBD. In embodiments, the extract comprises less than 0.15% THC. In embodiments, the extract further comprises up to 1% CBDV. In further embodiments, the highly purified extract comprises no more than 0.15% CBDA and/or no more than 0.5% CBD-C4. Alternatively, the CBD is present as a synthetic compound.

In embodiments, the dose of CBD is below 50 mg/kg/day. In embodiments, the dose of CBD is below 30 mg/kg/day. In embodiment, the dose of CBD is 20 mg/kg/day or greater, or is 10 mg/kg/day or greater.

In embodiments, the chewing gum composition for treating seizures in addition to containing one or more cannabinoids (e.g., CBD), also comprises one or more AEDs. In other embodiments, the method of treating seizures comprises administering a chewing gum composition comprising one or more cannabinoids and separately administering one or more AEDs. When separately administered from the chewing gum composition comprising one or more cannabinoids, the AED can also be administered in a second chewing gum composition, or can be administered by other standard routes of administration for the AED, including for example orally by administration of a tablet or capsule. In the case of separate administration of the chewing gum comprising a cannabinoid and one or more AEDs, the chewing gum and the AED composition can be administered simultaneously or sequentially.

Methods of Treating Tremors

In certain embodiments the chewing gum composition is used in the treatment of tremors. Tremor is an involuntary, rhythmic muscle contraction leading to shaking movements in one or more parts of the body including, but not limited to, the hands, arms, head, vocal cords, torso, and legs. Tremor may be intermittent or constant. In embodiments, the tremor is associated with certain neurological diseases or disorders. In embodiments, the neurological disease or disorder is multiple sclerosis, stroke, traumatic brain injury, Alzheimer's disease, Parkinson's disease, Huntington chorea; amyotrophic lateral sclerosis (ALS); Tourette syndrome; Pick's disease; multiple system atrophy (MSA), dementia with Lewy bodies (DLB), progressive supranuclear palsy (PSP), and corticobasal ganglionic degeneration (CBGD). Parkinson's disease being one often associated with tremor. In embodiments the tremor treated by the chewing gum composition described herein is essential tremor; dystonic tremor, cerebellar tremor, psychogenic tremor, or physiologic tremor. In embodiments, the tremor is associated with a hyperkinetic movement disorder (HMD) and disease, including but not limited to, chorea, dystonia, athetosis, myoclonus, stereotypies, tics, and hemiballismus.

Treatment of tremor is accomplished by administering the chewing gum composition disclosed herein to a subject in need thereof. The term treatment is used in its broadest sense and, as used herein, means lessening the severity, lessening the frequency, or otherwise alleviating tremor.

The method of treating tremor comprises chewing one or two pieces of gum comprising one or more cannabinoids as described herein. In embodiments, the chewing gum composition further comprises one or more anti-tremor drugs as described herein. Typically, up to two pieces of the chewing gum composition may be chewed at one time. Cannabinoids and derivatives thereof and anti-tremor drugs in these chewing gums according to embodiments may be released in a controlled manner and absorbed by a subject via transmucosal delivery mechanism. A mammal, such as a human being, may chew the chewing gum composition according to embodiments described herein 1 to 6 times a day to reduce the frequency of tremors and/or reduce the severity of tremors.

In embodiments, the chewing gum composition for the treatment of tremors, including those associated with certain neurological diseases or disorders, comprises one or more cannabinoids. In embodiments, the cannabinoid comprises, consists of, or consists essentially of CBD. In embodiments, the chewing gum composition for the treatment of tremors contains as an active ingredient essentially pure cannabinoids (whether synthetic or isolated from an extract). The cannabinoids may act, e.g., by direct antagonism of the NMDA receptor or by reducing the influx of calcium ions into the cell by another means such as binding with cannabinoid receptors. In embodiments, the one or more cannabinoids comprise, consist of, or consist essentially of CBD and THC. The amounts of cannabinoids in the chewing gum composition for treating tremors are the amounts for the chewing gum compositions disclosed herein. In embodiments, the chewing gum composition alleviates additional symptoms of the neurological disease or disorder. In embodiments, the chewing gum composition for the treatment of tremors further comprises one or more anti-tremor drugs including, but not limited to the AEDs and anti-tremor drugs disclosed herein.

In other embodiments, the method of treating tremor comprises administering a chewing gum composition comprising one or more cannabinoids and separately administering one or more anti-tremor drugs. When separately administered from the chewing gum composition comprising one or more cannabinoids, the anti-tremor drug(s) can also be administered in a second chewing gum composition, or can be administered by other standard routes of administration for the anti-tremor drug, including for example orally by administration of a tablet or capsule. In the case of separate administration of the chewing gum comprising a cannabinoid and one or more anti-tremor drugs, the chewing gum and the anti-tremor drug composition can be administered simultaneously or sequentially.

Subject in Need Thereof

In embodiments, a subject in need thereof is any person that suffers from symptoms (e.g., seizures or tremors). In embodiments, the seizures are associated with, and/or the subject has been diagnosed as having, epilepsy, including for example Lennox-Gastaut syndrome. In other embodiments, a subject in need thereof is any person that suffers from symptoms (e.g., tremors) associated with, and/or has been diagnosed as having, a neurological disease or disorder, including for example multiple sclerosis, stroke, traumatic brain injury, Alzheimer's disease, or Parkinson's disease.

Manufacture of the Gum Compositions

The chewing gum compositions described herein comprise a gum base and one or more active agents, such as one or more cannabinoids, and in embodiments, one or more AEDs or one or more anti-tremor agents.

In an embodiment, the method for manufacture of the chewing gum is exemplified in U.S. Pat. No. 9,744,128, incorporated herein by reference. In general the process for preparing the chewing gum compositions comprises placing one or more gum base(s) in a container with a sugar alcohol and heating the gum base(s) and one or more sugar alcohols to an internally-measured temperature between 140-160° F. to melt the gum base(s). The gum base may contain one or more elastomers, resins, softening agents, emulsifiers and/or fillers. The ingredients, including one or more active ingredients, sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients (e.g., one or more lubricants/glidants) in an orbital or planetary mixer, and tableted using a tablet press. This process preserves the efficacy of the active ingredient or ingredients by avoiding exposure to extreme temperatures, particularly during the mixing and milling.

In embodiments, other gum base compositions may be applied. The gum base can be prepared via application of a traditional mixing that utilizes mechanical actions while heating the mixture.

In an embodiment, the mixing pot is be preheated to approximately 115-125° C. After 10 minutes of preheating the elastomer, filler and additional softening ingredients are added to a mixing pot. The rubber along with the resin is mixed until the mixture is consistent. The softening ingredients can be added after about 30-50 minutes and mixed with the rubber and resin until an even consistency is achieved. The mixture can then be released into another pot to cool down to approximately 18-25° C.

In an embodiment, the mixing pot is preheated to approximately 55° C. After 10 minutes of preheating the gum base and filler are mixed in a mixing pot. After the mixture is consistent, additional ingredients can be added. Typically, the active agent is added in the first half of the mixing process.

Chewing Gum Compositions

The present disclosure provides a chewing gum composition comprising a gum base and one or more cannabinoids along with additional excipients. The chewing gum compositions can additionally include a sugar, a sugar blend, a sugar alcohol, a blend of sugar alcohols or combinations thereof, as well as additional excipients such as coloring agents, flavoring agents, lubricants and powder flow agents. In further embodiments, the composition comprises one or more sugar substitutes and flavoring agents. Flavors can include, for example, mint, peppermint, citrus, mixed berries, spearmint, wintergreen, and combinations thereof.

In embodiments, the chewing gum composition comprises, in addition to the active agent(s), one or more sugar alcohols, a gum base, one or more flavoring agents, one or more sweeteners, and tableting excipients. In further embodiments, the composition comprises about 45-65% sugar alcohols, about 15-35% gum base, about 2-15% flavoring agents and/or sweeteners, and about 1-10% tableting excipients. In embodiments, the composition comprises about 55% sugar alcohols, about 27% gum base, about 9% flavoring agents and/or sweeteners, and about 3% tableting excipients.

In an embodiment, the chewing gum composition comprises about 5% to about 80% of a gum base; about 10% to about 80% by weight of a sugar, a sugar blend, sugar alcohol, blend of sugar alcohols, sweetener, or combinations thereof; about 1% to about 20% by weight of a flavoring agent; about 0.1% to about 10% by weight of a lubricant or powder flow agent, or combinations thereof. In an embodiment, the chewing gum composition comprises about 55% to about 75% of a sugar, a sugar blend, sugar alcohol, blend of sugar alcohols, sweetener, or combinations thereof, about 20% to about 30% of a gum base, about 1% to about 15% of a flavoring agent or agents, about 0.1% to about 5% tableting lubricants and powder flow agents; and about 0.01% to about 2% by weight of one or more sugar substitutes.

Gum Base

The gum base imparts the chewing characteristics to the final gum composition. The gum base can also impact the release profile of the active ingredients, flavors and sweeteners. A suitable chewing gum base for use in the gum compositions described herein comprises one or more constituents including one or more elastomer, resin, plasticizer or softener, and filler. Certain components in the gum base may have more than one function in the composition. The gum base may also contain one or more stabilizing agents, coloring agents, and flavoring agents. In a preferred embodiment, the gum base comprises couma macrocarpa, glycerol esters of gum, microcrystalline wax, lecithin, and calcium carbonate.

In embodiments, the chewing gum composition comprises an elastomeric or natural chicle base as is commonly used in chewing gum formulations that are commercially available and accepted by the consumer. An elastomeric or natural chicle base is present in the chewing gum composition in an amount of about 10% to about 85% by weight, based on the total weight of the chewing gum composition.

Elastomers provide the rubbery, cohesive nature to the gum and may include natural or synthetic types. The elastomer may be any water-insoluble polymer including the natural and synthetic gum polymers utilized for chewing gum, for example those listed in the U.S. Code of Federal Regulations, Title 21, Section 172.615. Useful natural elastomers include natural rubber such as smoked or liquid latex and guayule, natural gums such as jelutong, couma macrocarpal (also called lechi caspi), perillo, sorva, massaranduba balata, massaranduba chocolate, nispero, rosidinha, chicle, gutta percha, gutta kataiu, niger gutta, tunu, chilte, chiquibul, gutta hang kang.

Synthetic elastomers include high-molecular weight elastomers such as butadiene-styrene copolymers, polyisobutadiene and isobutylene-isoprene copolymers, low-molecular weight elastomers such as polybutene, polybutadiene and polyisobutylene, vinyl polymeric elastomers such as polyvinyl acetate, polyethylene, vinyl copolymeric elastomers such as vinyl acetate/vinyl laurate, vinyl acetate/vinyl stearate, ethylene/vinyl acetate, polyvinyl alcohol or mixtures thereof. Combinations of synthetic elastomer in the gum base can include a synthetic elastomer having a high-molecular weight and a low-molecular-weight elastomer. Combinations of synthetic elastomers include, but are not limited to, polyisobutylene and styrene-butadiene, polyisobutylene and polyisoprene, polyisobutylene and isobutylene-isoprene copolymer (butyl rubber) and a combination of polyisobutylene, styrene-butadiene copolymer and isobutylene isoprene copolymer, and all of the above individual synthetic polymers in admixture with polyvinyl acetate, vinyl acetate-vinyl laurate copolymers, respectively and mixtures thereof.

Resins provide a cohesive body or strength to the gum composition and may also act as softeners and include glycerol esters of gum, terpene resins, and/or polyvinyl acetate.

Plasticizers (softening agents) affect the firmness of the gum base. Elastomer plasticizers include natural rosin esters often referred to as ester gums. Such plasticizers include methyl, glycerol and pentaerythritol esters of rosins and modified rosins, such as hydrogenated, dimerized and polymerized rosins. Examples of plasticizers include glycerol ester of wood and gum rosin, glycerol ester of partially hydrogenated wood and gum rosin, glycerol ester of polymerized wood and gum rosin, glycerol ester of partially dimerized wood and gum rosin, glycerol ester of tall oil rosin, pentaerythritol ester of wood and gum rosin, pentaerythritol esters of partially and fully hydrogenated wood and gum rosin, methyl esters of wood and gum rosins and partially and fully hydrogenated methyl esters of wood and gum rosin. Synthetic plasticizers include terpene resins derived from α-pinene, β-pinene and/or dipentene.

Emulsifying agents may act as softeners and can also help to hydrate the composition. In embodiments, the emulsifier is lecithin and/or glycerol monostearate. Emulsifiers include monoglycerides, diglycerides, acetylated mono and diglycerides, distilled mono- and diglycerides, glycerol monostearate, propylene glycol monostearate, Na-, K-, Mg- and Ca-stearates, glycerol triacetate, fatty acid monoglycerides (e.g., stearic, palmitic, oleic and linoleic acids), lactic acid esters and acetic acid esters of mono- and diglycerides, sugar esters of edible fatty acids, lecithin and hydroxylated lecithin.

One or more fillers may be used in the chewing gum compositions to modify the texture and aid in processing. In embodiments, the filler comprises talc and/or calcium carbonate. Fillers include celluloses and cellulose derivatives including microcrystalline cellulose, hydroxypropylcellulose and sodium carboxymethylcellulose, lactose, starches including potato starch and corn starch, carbohydrates including a cellulose derivative, e.g. hemicellulose. The cellulose derivative may be of natural origin, e.g. dextran, agarose, agar, pectin, alginate, xanthan, chitosan, starch. The cellulose derivative may also be of synthetic or semi-synthetic origin. In certain embodiments, the filler comprises microcrystalline cellulose (MCC), bamboo fibers, or combinations thereof. Specific examples of a suitable microcrystalline cellulose is microcrystalline cellulose comprising: AVICEL™ grades PH-100, PH-102, PH-103, PH-105, PH-112, PH-113, PH-200, PH-300, PH-302, VIVACEL™ grades 101, 102, 12, 20 and EMOCEL™ grades 50M and 90M, and the like, and mixtures thereof. The fillers may be present in the composition from about 5% to about 50% by weight, based on total weight of the composition.

Sweeteners

Sweeteners may be used in the chewing gum compositions to impart a sweet taste to the gum composition. In addition, sweeteners can act a softener and/or as a filler in the gum composition. Suitable sweeteners include one or more of a sugar, sugar blend, sugar alcohol, a blend of sugar alcohols, high intensity sweeteners, sugar substitutes, or combinations thereof. Sugar substitutes include sucralose, monk fruit, neotame, licorice extract, stevia, honey or agave nectar, or combinations thereof. Suitable high intensity sweeteners include sucralose, stevia, honey, monk fruit, neotame, licorice extract, agave nectar, or combinations thereof. Sugar alcohols include sorbitol, isomalt, xylitol, maltitol, mannitol, erythritol or combinations thereof. Sugars include dextrose, sucrose, fructose, glucose or combinations thereof. The amount of sweetener in the gum composition depends on factors such as potency of the sweetener, rate of release, desired sweetness of the product, and amount and type of flavor used.

Flavorings

Natural and/or artificial flavorings may be used in the gum compositions. Both synthetic flavoring agents and natural flavoring agents derived from plants, leaves, flowers, fruits, etc. and combinations thereof can be used. Examples of flavoring agents include spearmint oil, cinnamon oil, oil of wintergreen (methysalicylate) and peppermint oils. Synthetic and natural fruit flavors useful as flavoring agents include citrus oil, e.g., lemon, orange, lime and grapefruit; fruit essences including apple, strawberry, cherry, banana, pineapple; and other flavorings such as aldehydes and esters including cinnamyl acetate, cinnamaldehyde, citral diethyl acetal, dihydrocavryl acetate, eugenyl formate, p-methylamisol, and others. Flavoring agents are generally liquids. However, they can also be used as spray dried solids. The use of flavoring agents having other physical forms such as powdered flavorings, beaded flavorings and encapsulated flavorings may also be used in the gum compositions described herein. Preferred flavoring agents in the chewing gum compositions described herein include mint, peppermint, citrus, mixed berries, spearmint, wintergreen, and combinations thereof.

The amount of flavoring agent employed is determined by flavor type and the strength of flavor desired. In embodiments, flavoring amounts of about 0.05% to about 3.0% by weight of the overall chewing gum composition are used, preferably about 0.3% to about 1.5%, more preferably about 0.7% to about 1.2% by weight.

In embodiments, additional flavoring agents are added to the chewing gum composition granules immediately prior to tableting. Preferably, the additional flavoring agent is in a dry form, e.g., spray dried flavoring or encapsulated flavoring. However, liquid flavoring can be added if it is first blended with the dry components of the lubricating system.

Lubricants and Powder Flow Agents

Lubricants and powder flow agents (e.g., glidants) may be used in the chewing gum compositions to aid processing. Tableting lubricants and powder flow agents include magnesium stearate, silicon dioxide, calcium stearate, stearic acid, talc, rice bran-based excipients, or combinations thereof.

Binders

Binders include water-soluble synthetic polymer, polyvinlypyrrolidone (povidone), sorbitol, mannitol, xylitol, lactitol, erythritol, pregelatinized starch, modified starch (e.g., starch sodium octenylsuccinate), hydroxypropylmethylcellulose and others.

Stabilizing Agents

In embodiments, the gum compositions contain one or more stabilizing agent to prolong the shelf life of the active ingredient(s) and/or other components of the gum composition. In embodiments, the stabilizing agent is an antioxidant. Suitable antioxidants include butylated hydroxyanisole (BHA), butylated hydroxytoluene (BHT), betacarotenes, tocopherols, acidulants such as vitamin C, propyl gallate, and combinations thereof.

All references referred to herein are incorporated in their entirety.

### EXAMPLES

Preparation of a Chewing Gum Containing CBD and AED:

A chewing gum composition having 40 mg of CBD and 200 mg of AED (gabapertin) is prepared. In general the process for preparing the chewing gum composition comprises placing the components of the gum base in a container with a sugar alcohol and heating in an oven to an internally-measured temperature between 140-160° F. to melt the gum base. The ingredients, including the active ingredients, sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients in an orbital or planetary mixer, and tableted.

TABLE 1
Formulation of chewing gum

| Ingredient | wt % in gum |
|---|---|
| CBD (encapsulated) | 2 |
| Gabapentin (encapsulated) | 10 |
| Gum Base | 47.6 |
| Sugar Alcohol (xylitol) | 30 |
| Glycerine | 4.5 |
| Sacharrine | 0.4 |
| Water | 1.5 |
| Citric Acid | 0.5 |
| Cellulose | 0.5 |
| Peppermint aroma oil | 1.5 |
| Pepperment powder | 1.5 |

1. A composition comprising one or more cannabinoids, one or more anti-epileptic drugs (AEDs) and a gum base.

2. The composition according to claim 1, wherein the one or more AEDs is selected from the group consisting of rufinamide, lamotrigine, felbamate, clobazam and topiramate, carbamezapine, gabapentin, oxcarbazepine, pregabalin, tiagabineor and vigabatrin, acetazolamide, brivaracetam, clonazepam, eslicarbazepine acetate, ethosuximide, everolimus, lacosamide, lamotrigine, levetiracetam, oxcarbazepine, perampanel, phenobarbital, phenytoin, piracetam, pregabalin, primidone, rufinamide, sodium valproate, stiripentol, tiagabine, topiramate, valproic acid, vigabatrin, zonisamide or combinations thereof.

3. The composition of claim 2, wherein the AED is gabapentin.

4. A composition comprising one or more cannabinoids, one or more anti-tremor drugs and a gum base.

5. The composition of claim 4, wherein the one or more anti-tremor drugs is a beta blocker, a benzodiazepine, or combinations thereof.

6. The composition of claim 4, wherein the one or more anti-tremor drugs is selected from the group consisting of propranolol, atenolol, metoprolol, nadolol, sotalol, alprazolam, clonazepam, diazepam, and lorazepam, or combinations thereof.

7. The composition according to claim 1, wherein the one or more cannabinoids is selected from the group consisting of cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof.

8. The composition of claim 7, wherein the one or more cannabinoids is CBD.

9. The composition according to claim 1, wherein the gum base comprises one or more elastomers, resins, softening agents, emulsifiers and fillers.

10. The composition according to claim 1, wherein the gum base comprises couma macrocarpa, glycerol esters of gum, microcrystalline wax, lecithin and calcium carbonate.

11. The composition according to claim 1, further comprising one or more sugar alcohols.

12. The composition of claim 11, wherein the one or more sugar alcohols comprises one or more of maltitol, sorbitol, isomalt, and xylitol.

13. The composition according to claim 11, further comprising one or more additional sweeteners.

14. The composition of claim 13, wherein the one or more additional sweeteners are selected from a sugar, a high intensity sweetener, a sugar substitute, or a combination thereof.

15. The composition according to claim 1, further comprising one or more flavoring agents, and one or more lubricants and powder flow agents.

16. The composition according to claim 1, comprising maltitol, sorbitol, isomalt, xylitol, Stevia, a flavoring agent, magnesium stearate, and silicon dioxide.

17. A method of treating seizures comprising administering to a subject in need thereof a composition according to claim 1.

18. The method according to claim 17, wherein the seizures are associated with epilepsy.

19. The method according to claim 17, wherein the seizures are associated with Lennox-Gastaut syndrome (LGS), Dravet syndrome, self-limited, late-onset, occipital epilepsy (Gastaut syndrome), epilepsy with eyelid myoclonia (Jeavons syndrome), temporal lobe epilepsy (TLE), juvenile absence epilepsy (JAE) or juvenile myoclonic epilepsy (JME).

20. The method according to claim 17, wherein the subject has not responded to one or more AED drugs.

21. The method according to claim 17, wherein administration of the chewing gum composition results in a reduction in the frequency of drop seizures and/or reduction in the total number of seizures experienced by the subject.

22. A method of treating tremor comprising administering to a subject in need thereof a composition according to claim 1.

23. The method of claim 22, wherein the tremor is associated with a neurological disease or disorder.

24. The method of claim 23, wherein the neurological disease or disorder is multiple sclerosis, stroke, traumatic brain injury, Alzheimer's disease, Parkinson's disease, Huntington chorea; amyotrophic lateral sclerosis (ALS); Tourette syndrome; Pick's disease; multiple system atrophy (MSA), dementia with Lewy bodies (DLB), progressive supranuclear palsy (PSP), or corticobasal ganglionic degeneration (CBGD).

25. The method of claim 22, wherein the tremor is essential tremor; dystonic tremor, cerebellar tremor, psychogenic tremor, or physiologic tremor.

26. The method of claim 22, wherein the tremor is associated with a hyperkinetic movement disorder selected from the group consisting of chorea, dystonia, athetosis, myoclonus, stereotypies, tics, and hemiballismus.

27. The method according to claim 22, wherein the one or more cannabinoids further comprises THC.

28. The composition of claim 1, wherein the composition is obtained by a method comprising the steps of:
(i) placing the gum bases in a container with a sugar alcohol and heating to an internally-measured temperature of between 140-160° F. to melt the gum base;
(ii) combining the active agents, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).
